SEQUENCE LISTING (20 sequences)

SEQ ID NO: 1 - DNA, 30 bp, artificial sequence
  mcks1-u primer for mouse Cks1 cDNA
ctcctgcagagcgatcatgtcgcacaaaca

SEQ ID NO: 2 - DNA, 30 bp, artificial sequence
  mcks1-d primer for mouse Cks1 cDNA
cttctgcagcttcatttctttggtttcttg

SEQ ID NO: 3 - DNA, 27 bp, artificial sequence
  mcks2-u primer for mouse Cks1 cDNA
tttctgcagcgcgccggcaggatggcc

SEQ ID NO: 4 - DNA, 30 bp, artificial sequence
  mcks2-d primer for mouse Cks1 cDNA
agataagcttcatttttgttgttcttttgg

SEQ ID NO: 5 - DNA, 65 bp, artificial sequence
  pRcks1-t primer for rat Cks1-shRNA cDNA
gatccgtcagaagcatgtgaaatcttcaagagagatttcacatgcttctgacttttttacgcgtg

SEQ ID NO: 6 - DNA, 65 bp, artificial sequence
  pRcks1-b primer for rat Cks1-shRNA cDNA
aattcacgcgtaaaaaagtcagaagcatgtgaaatctctcttgaagatttcacatgcttctgacg

SEQ ID NO: 7 - DNA, 65 bp, artificial sequence
  pRcks2-t primer for rat Cks2-shRNA cDNA
gatccgccagaaccgcatattcttttcaagagaaagaatatgcggttctggcttttttacgcgtg

SEQ ID NO: 8 - DNA, 65 bp, artificial sequence
  pRcks2-b primer for rat Cks2-shRNA cDNA
aattcacgcgtaaaaaagccagaaccgcatattctttctcttgaaaagaatatgcggttctggcg

SEQ ID NO: 9 - DNA, 65 bp, artificial sequence
  pMcks1-t primer for mouse Cks1-shRNA
gatccggtccactatatgatccatttcaagagaatggatcatatagtggaccttttttacgcgtg

SEQ ID NO: 10 - DNA, 65 bp, artificial sequence
  pMcks1-b primer for mouse Cks1-shRNA
aattcacgcgtaaaaaaggtccactatatgatccattctcttgaaatggatcatatagtggaccg

SEQ ID NO: 11 - DNA, 65 bp, artificial sequence
  pMcks2-t primer for mouse Cks2-shRNA
gatccgtccaacagagtctaggatttcaagagaatcctagactctgttggacttttttacgcgtg

SEQ ID NO: 12 - DNA, 65 bp, artificial sequence
  pMcks2-b primer for mouse Cks2-shRNA
aattcacgcgtaaaaaagtccaacagagtctaggattctcttgaaatcctagactctgttggacg

SEQ ID NO: 13 - DNA, 28 bp, artificial sequence
  rcks1-u primer for rat Cks1 cDNA
ctggatccgcaaattcacaccatccctg

SEQ ID NO: 14 - DNA, 28 bp, artificial sequence
  rcks1-d primer for rat Cks1 cDNA
gtgatatcctgatagcgtgaccgtgttg

SEQ ID NO: 15 - DNA, 19 bp, artificial sequence
  rcks2-u primer for rat Cks2 cDNA
cgtttccttgtcccggttt

SEQ ID NO: 16 - DNA, 20 bp, artificial sequence
  rcks2-d primer for rat Cks2 cDNA
tatgcggttctggctcatgg

SEQ ID NO: 17 - DNA, 30 bp, artificial sequence; identical to SEQ ID NO: 1
  hcks1-u primer for human Cks1 cDNA

SEQ ID NO: 18 - DNA, 30 bp, artificial sequence; identical to SEQ ID NO: 2
  hcks1-d primer for human Cks1 cDNA

SEQ ID NO: 19 - DNA, 27 bp, artificial sequence
  hcks2-u primer for human Cks2 cDNA
tttctgcagcgcgccggcaggatggcc

SEQ ID NO: 20 - DNA, 30 bp, artificial sequence; identical to SEQ ID NO: 4
  hcks2-d primer for human Cks2 cDNA

(19) United States
(12) Patent Application Publication
(10) Pub. No.: US 2009/0285800 A1
(43) Pub. Date: Nov. 19, 2009

(54) Aberrant expression of CKS1 and CKS2 and uses thereof

(76) Inventors: Fen Wang, Bellaire, TX (US); Yongshen Lan, Houston, TX (US)

Correspondence Address: Benjamin Aaron Adler;ADLER & ASSOCIATES, 8011 Candle Lane, Houston, TX, 77071, US

(21) Appl. No.: 12/454,433

(22) Filed: May 18, 2009

Related U.S. Application Data
(60) Provisional application No. 61/127,924, filed on May 16, 2008.

(51) Int. Cl. A61K 39/395; C12Q 1/68; A61K 38/16; A61K 31/7088

(52) U.S. Cl. 424/130.1; 435/6; 514/8; 514/44 A

(57) ABSTRACT

The present invention is drawn to providing a therapeutic strategy for prostate cancer. In this regard, the present invention demonstrates that elevated expression of Cks1 contributes to tumorigenicity of prostate tumor cells by promoting cell growth and elevated expression of Cks2 protects cells from apoptosis. Accordingly, the present invention discloses methods to diagnose and treat prostate cancer by targeting expression of Cks1 and Cks2 proteins.

### CROSS-REFERENCE TO RELATED APPLICATIONS

This non-provisional applications claims benefit of priority under 35 U.S.C. §119(e) of provisional U.S. Ser. No. 61/127,924, filed May 16, 2008, now abandoned, the entirety of which is hereby incorporated by reference.

### FEDERAL FUNDING LEGEND

This invention was made with government support under CA096824 awarded by NCI and DAMD17-03-1-0014 awarded by DOD. The government has certain rights in the invention.

### BACKGROUND OF THE INVENTION

1. Field of the Invention

The present invention relates to the fields of cell signaling pertaining to tumor cell growth, angiogenesis, and metastasis. More specifically, the present invention discloses decreased expression of Sp family proteins by curcumin and several polycyclic steroidal/triterpenoid-like and related compounds resulting in inhibition of growth and proliferation, angiogenesis and metastasis of cancer cells.

2. Description of the Related Art

The human Cks (also designated as CksHs) family consists of two well-conserved members, Cks1 and Cks2, both of which are identified based on the sequence homology to yeast suc1 and Cks1 (Cdc28 kinase subunit 1) that are essential for cell cycle control (1-3). Emerging evidence shows that the two Cks members in mammalian cells have distinct regulatory function from the yeast counterparts. Cks1 is required for SCF^{Skp2}-mediated ubiquitination and degradation of p27^{kip1}, which is essential for the G1/S transition during the cell cycle (4-5). Although the function of Cks2 in the cell cycle is not clear, expression of both Cks1 and Cks2 has been shown to oscillate during the cell cycle and is positively related to cell proliferation (6). Recently, Cks2 has been shown essential for the first metaphase/anaphase transition of mammalian meiosis (7).

Numerous reports demonstrate that expression of Cks2 is frequently elevated in tumors of different tissue origins, including nasopharyngeal carcinoma, melanocytic tumors, Wilms tumor, breast, bladder, cervical, esophageal, lymphoid, and metastatic colon cancer (6, 8-14). In addition, expression of Cks2 is downregulated by p53, a tumor suppressor, at the transcription and the protein levels (15). Similarly, elevated expression of Cks1 has been found in tumors from a variety of tissue origins, and is correlated with poor survival rate of oral squamous cell carcinoma (16-23). Knockdown of Cks1 inhibits growth and tumorigenicity of oral squamous cells (23). Consistent with the finding that Cks1 is a negative regulator of a cell cycle control protein, p27^{kiP1}, elevated expression of Cks1 is found coincident with the reduction of p27^{kiP1} proteins in tumor cells. Expression of p27^{kiP1} is often aberrantly reduced in cancer cells, including prostate cancer cells. We recently reported that the fibroblast growth factor (FGF) signaling axis directly regulates activity of Cks1 during the G1/S transition in the cell cycle through FGF receptor substrate 2α, a proximal FGF receptor-interactive adaptor protein of the FGF receptor tyrosine kinase, which connects multiple downstream signaling molecules to the FGF receptor tyrosine kinase (24).

The prostate is an accessory organ of the male reproductive system, which consists of epithelial and stromal compartments separated by basement membranes. Cancers arising from the prostate epithelium are the most commonly diagnosed cancer and the second most common cause of cancer death in American males. In America alone, about 230,000 new cases and 30,000 deaths are expected every year. To date, whether the Cks family is overexpressed in prostate tumor, and if yes, whether aberrantly expressed Cks contributes to prostate tumor initiation and progression remains to be characterized. Recently, it has been shown that Cks1 expression is associated with the aggressive behavior of prostate cancer (25). In addition, treating LNCaP prostate cancer cells with an herbal mixture, PC-SPES, inhibits cell proliferation and reduces expression of Cks2 (26).

Thus, the prior art is still deficient in cancer therapies employing the Cks family, which results in inhibition of growth, angiogenesis and metastasis of prostate cancer cells. The present invention fulfills this long-standing need and desire in the art.

### SUMMARY OF THE INVENTION

To determine whether overexpression of the Cks family was associated with prostate tumorigenesis, expression patterns of Cks1 and Cks2 were analyzed in the Dunning prostate tumor model of rats, the TRAMP prostate cancer model of mice, human prostate cancer cell lines, and human prostate cancer samples. The results obtained demonstrated that all tested human, rat, and mouse prostate tumor tissues and cells exhibited elevated expression of Cks1 and Cks2 in comparison with normal prostate tissues that only weakly express Cks1 and Cks2. Further, forced expression of Cks1 and Cks2 by transfection in benign prostate tumor cells promoted cell population increases. Consistently, knockdown of Cks1 and Cks2 expression by shRNA in malignant AT3 cells inhibited cell population growth. In addition, knockdown of Cks1 expression also inhibited anchorage-independent growth, and migration activities, and knockdown of Cks2 expression induced the cells to undergo massive program cell death, especially when the cells were maintained in suboptimal growth condition. The results suggested that elevated expressed Cks1 may contribute to prostate tumorigenesis by promoting proliferation, anchorage-independent growth, and migration of the cells, and Cks2 by protecting cells from undergoing programmed cell death. The findings discussed herein provide for cancer therapeutic strategies based on inhibition of the Cks activity.

The present invention is directed to a method for treating prostate cancer in an individual in need of such treatment. Such a method comprises the step of administering pharmaceutically effective amounts of a compound that downregulates the expression of Cks1 gene, Cks2 gene or both to the individual, thereby treating the prostate cancer in the individual.

The present invention is also directed to a method for treating prostate cancer in an individual in need of such treatment. Such a method comprises the step of: administering pharmaceutically effective amounts of a compound that downregulates the expression of Cks1 gene product, Cks2 gene product or both to the individual, thereby treating the prostate cancer in the individual.

The present invention is further directed to a method for diagnosing prostate cancer in an individual. This method comprises obtaining a biological sample from the individual; and determining the expression level of the Cks1 gene, the Cks1 gene product, the Cks2 gene, the Cks2 gene product or a combination thereof in the sample. Overexpression of the Cks1 gene, the Cks1 gene product, the Cks2 gene, the Cks2 gene product or a combination thereof compared to expression level of the Cks1 gene, the Cks1 gene product, the Cks2 gene, the Cks2 gene product or a combination thereof in the sample from a control individual indicates that the individual has prostate cancer.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1F show overexpression of Cks1 and Cks2 in prostate tumors. In FIGS. 1A-1C, total RNA samples were extracted from the indicated tumors and cells. Expression of Cks1 and Cks2 was assessed with RNase protection assays or RT-PCR. Beta-actin was used as a loading control. Vertical lines indicate that the lane orders had been rearranged for better illustrations. Specific bands were quantified using the Image J software, and the values were expressed as abundances relative to beta-actin (C/A). In FIG. 1D, total RNA samples were prepared from human prostate tumors (Tumor) and the adjacent normal tissues (Normal). The expression of Cks2 was assessed with real-time RT-PCR. Data were normalized to beta-actin and were expressed as ratios of Cks2/b-actin×10³. In FIG. 1E, human prostate sections with tumor and normal histological structures were prepared for in situ hybridization with probes specific for human Cks2. Cks2 expression was apparent in tumor foci but absent in normal section. Arrow heads indicate the epithelium. In FIG. 1F, prostate tissue sections were prepared from TRAMP and control mice at the age of 6 weeks, and expression of Cks1 (panels a, c, and e) and Cks2 (panels b, d, and f) was assessed by in situ hybridization. Panels a, b, areas with relatively normal prostate tissue structures; Panels c, d, areas with high-grade PIN lesions; Panels e and f, areas with carcinoma lesions. Arrows indicate Cks expression in lesion foci. A, AT3 cells; E, DTE cells; S, DTS cells; AT, AT tumor; DT, tumors derived from DTE cells; D3, DT3 tumors; NP, normal prostates; C2, TRAMP C2 cells; T, TRAMP tumors; RT, reverse transcription; −, negative control without RNA samples.

FIGS. 2A-2J show that aberrantly expressed Cks1 and Cks2 contribute to prostate cancer cell growth. In FIGS. 2A-2C, AT3 cells stably transfected with Cks1-, Cks2-, and stochastic-shRNA were inoculated in 24-well dishes with or without the doxycycline induction and the cell numbers were determined daily. Note that Cks2 shRNA-expressing cells were cultured in 50% conditioned medium as described. The data are means±SD of triplicate samples. In FIGS. 2D-2F, RT-PCR analyses of Cks expression for FIGS. 2A-2C, respectively, are shown. GADPH (FIG. 2D) or beta-actin (FIGS. 2E-2F) was used as a loading control. I, induced with doxycycline; C, without doxycycline induction; N, untransfected cells. In FIG. 2G, lysates were prepared from the transfected AT3 cells with or without doxycycline induction were Western blotted with anti-p27^{kiP1} or anti-b-actin antibodies. The specifically bound antibodies were visualized with ECL-Plus chemoluminescent reagents. In FIG. 2H, pool of DTE cells stably expressing Cks1-GFP and Cks2-GFP fusion proteins, or GFP alone, were cultured in 24-well plates in 2.5% fetal bovine serum and the cells numbers were determined daily. The data are means±sd of triplicate samples. In FIG. 2I, RT-PCR analyses of Cks expression for FIG. 2H is shown. Beta-actin and GFP were used as a loading control. In FIG. 2J, lysates prepared from the transfected DTE cells with or without doxycycline induction were Western blotted with anti-p27^{kip1} or anti-betaactin antibodies. The specifically bound fractions were visualized with alkaline phosphatase staining.

FIGS. 3A-3D show that knockdown of Cks1 and Cks2 expression has different impacts on malignant AT3 cells. In FIG. 3A, stable Cks1 and Cks2 shRNA transfectants of AT3 cells were inoculated in soft agar plates with or without doxycycline induction as indicated. Colony numbers were scored 10 days after the inoculation and expressed as means±sd of duplicate samples. In FIG. 3B, the same cells were cultured in Transwell inserts. Only the medium in outer chambers contained 10% FBS. After incubation at 37 C for 12 hours, the numbers of cells migrating across the membranes were determined. Data are means±sd of triplicate samples. In FIG. 3C, the same cells cultured in 6-well plates were treated with doxycycline to induce shRNA expression. Cell morphological changes were documented at 48 hours after the induction by staining with 0.5% Methylene Green. FIG. 3D shows statistical analyses of morphological changed cells induced by Cks2 knockdown. Data are means±sd of triplicate samples. N, not induced; I, induced.

FIGS. 4A-4C show that knockdown of Cks2 expression in AT3 cells induces apoptosis. In FIG. 4A, stable Cks1- and Cks2-shRNA transfected AT3 cells were cultured in 6-well dishes. Expression of the shRNAs was induced by doxycycline induction. The apoptotic cells were stained with ApoPcentage dye and were visualized by microscopy. In FIGS. 4B-4C. The same cells were cultured in 15-cm dishes, and the expression of shRNAs was induced with doxycycline for 48 hours. The caspase 3 activity was quantitated as described above (FIG. 4B). Data are expressed as fold of increase from non-induced cells and are means±SD of triplicate samples. Expression of Bad, Bcl-xl, and Akt was assessed with Western blot analyzed. In FIG. 4C, beta-actin was used as loading controls. I, induced; N, not induced.

FIGS. 5A-5B show that knockdown of Cks2 expression in TRAMP C2 cells induces programmed cell death. In FIG. 5A, mouse Cks2-shRNA and GFP were coexpressed in TRAMP C2 cells by transient transfection. The apoptotic cells were stained with Cy3-conjugated Annexin V at day 2 after the transfection. The GFP expression and Annexin V-Cy3 stained cells were visualized by fluorescent microscopy and the cell morphology by phase contrast microscopy. In FIG. 5B, The GFP- and Annexin V/GFP-positive cells were quantified. The data are the percent of Annexin V/GFP double positive cells in total GFP-positive pool and are means±SD of triplicate samples.

FIGS. 6A-6B show that knockdown of Cks2 expression inhibits tumorigenicity of TRAMP tumor cells. In FIG. 6A, stable transfectants of TRAMP C2 cells (1×10⁶) carrying inducible expressed Cks2-shRNA or control shRNA sequences were implanted subcutaneously in BL6/C57 mice as described³³. Expression of shRNA was induced with doxycycline in drinking water where indicated. Mice were observed every day and palpable tumors were recorded. The percentage of tumor-free mice was scored once a day. The y-axis is the number of tumor-free mice at the respective age, as a percentage of all mice in each group. In FIG. 6B, expression of Cks2 in 2 individual stable Cks2-shRNA transfected clones was assessed with RT-PCR. b-actin was used as loading controls. C, control cells carrying stochastic shRNA sequence; Cks2, cells carrying Cks2-shRNA; DOX, doxycycline.

FIG. 7 shows that Cks2 expression in prostate cancer cells was not changed in serum starved cells. The indicated cells were cultured in serum-free medium for 48 hour before being harvested for RNA extraction. Expression of Cks1 and Cks2 was assessed with RT-PCR (39 cycles) as described in the methods. Beta-actin (20 cycles) was used as internal loading controls, and no RT was used as negative controls.

### DETAILED DESCRIPTION OF THE INVENTION

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “effective amount” or “pharmaceutically effective amount” are interchangeable and refer to an amount that results in an antiproliferative effect against cancer cells, e.g., prostate cancer cells, in vitro or an improvement or remediation in the cancer in vivo. Those of skill in the art understand that the effective amount may improve the patient's or subject's condition, but may not be a complete cure of the cancer.

As used herein, the term “individual” refers to any target of the treatment.

As used herein, the term “treating” or the phrase “treating a prostate cancer” includes, but is not limited to, halting the growth of the cancer, killing the cancer, or reducing the size of the cancer. Halting the growth refers to halting any increase in the size, or the number of, or size of the cancer cells, or to halting the division of the neoplasm, or the division of cancer cells. Reducing the size refers to reducing the size of the cancer, or the number of cancer cells, or the size of the cancer cells.

The present invention demonstrates that expression of Cks1 and Cks2 was elevated in prostate tumors, the Dunning prostate tumors in rat, the TRAMP tumors in mouse, and more importantly, in human prostate tumors and cell lines. Forced expression of Cks1 and Cks2 in benign prostate tumor epithelial cells accelerated cell growth and knockdown of Cks1 and Cks2 malignant prostate tumor cells inhibited cell growth, anchorage-independent growth, and migration activity. Additionally, knockdown of Cks2 expression induced apoptosis in vitro and compromised tumorigenic activity of the cells in vivo. These results suggest that elevated expression of Cks1 may contribute to tumorigenicity of prostate tumor cells by promoting cell growth and elevated expression of Cks2 by protecting the cells from undergoing apoptosis.

The function of the Cks family appears to be complicated. In yeast, both Cks1 and Cks2 bind to Cdk1 at a high affinity and function as a suppressor of Cdk1 mutants (1, 3, 34). Despite the high sequence homology between the two members, whether Cks1 and Cks2 in mammalian cells elicit redundant or synergistic activities is not well understood. Further, one report discloses that Cks1 compensates for Cks2 loss of function in the germ line (7). In mammalian cells, Cks1 induces an allosteric change in Skp2, which increases its affinity for phosphorylated p27^{kiP1} and triggers ubiquitination of p27^{kiP1}, resulting in a rapid proteasome-mediated degradation of p27^{kiP1} during G1/S transition in the cell cycle. To date, decreases in p27^{kiP1} expression or mutations of p27^{kiP1} have been reported in tumors of many tissue origins, including prostate cancer (35-36). The present invention demonstrates that forced expression of Cks1 reduced p27^{kiP1} in the cells and accelerated cell growth, while knockdown Cks1 expression in malignant AT3 cells inhibited proliferation and increased p27^{kiP1} abundance.

In addition to being a mitotic inhibitor, p27^{kiP1} also has cell cycle-independent function, including regulating cell migration. However, p27^{kiP1} has been shown to promote and inhibit cell migration (37-42). Recently, it has been shown that binding of p27^{kiP1} to the microtubule-destabilizing protein stathmin regulates the activity of p27^{kiP1} to repress cell migration. Upregulation of p27^{kiP1} or downregulation of stathmin expression results in inhibition of cell motility that is essential for invasion and metastasis of tumors (43-44). The present invention shows that knockdown of Cks1 expression increased p27^{kiP1} abundance in the cells and inhibited cell migration, which was consistent with the finding that p27^{kiP1} inhibits migration and invasion of tumor cells. In addition, inhibition of p27^{kiP1} in LNCaP cells significantly increases colony formation in soft agar, suggesting that p27^{kiP1} inhibits anchorage-independent growth activity of prostate cancer cells (45). Consistently, this data shows that knockdown of Cks1 expression increased abundance of p27^{kiP1} and inhibited colony formation activity of the malignant AT3 cells. The result further suggests that overexpression of Cks1 might contribute to the tumorigenicity through promoting anchorage-independent growth activity.

In contrast to Cks1, knockdown of Cks2 expression in AT3 cells did not increase p27^{kiP1} abundance. Instead, it slightly reduced the abundance of p27^{kiP1}. It is possible that the reduction of p27^{kiP1} abundance in AT3 cells was a result of cell death caused by knockdown of Cks2 expression. Similarly, forced expression of Cks2 did not reduce p27^{kiP1} albeit it accelerated DTE cell growth. Therefore, although changes in Cks2 expression affect cell growth and has been shown to oscillate during the cell cycle (6), it is likely that Cks2 may not directly control the cell cycle progression. Indeed, expression of Cks2 in DU145, AT3, and TRAMP C2 cells was not reduced when the cells were cultured in serum-free medium for 48 hours, in which condition, the cell proliferation was relatively lower than in full culture medium. It is possible that changes in Cks2 expression may impact cell growth by preventing apoptosis. Both cell growth and apoptosis assays showed that knockdown of Cks2 expression in malignant AT3 cells had a more potent impact in cells maintained in non-optimal culture conditions than in those maintained in optimal condition. This may explain why the leaky expression of Cks2 shRNA had limited effects on stock cell cultures that were maintained in the optimal conditions, yet, had significant effects on in vivo tumorigenesis analyses where the cells were implanted subcutaneously and did not have optimal supplies of oxygen and nutrition.

Together with the fact that Cks2 null mice did not have apparent defect in all tissues except the testes (7), the data here imply that Cks2 may not be essential for growth, differentiation, and maintenance of normal prostatic cells, yet, may be essential for protecting the cells from undergoing apoptosis in harsh growth conditions, such as deficient in nutritional or oxygen supplies. Thus, inhibition of Cks2 activity may be of therapeutic potential for tumor treatment, and promotion of Cks2 activity may be useful for preventing cells to undergo apoptosis due to insufficient oxygen or nutrition supplies, such as in stroke, heart attacks, or other injury. In addition, although having distinct roles, the two Cks members may synergistically contribute to prostate tumorigenesis by promoting cell proliferation and preventing apoptosis. Overall, the results discussed herein suggests that overexpressed Cks1 accelerates the population growth by directly promoting p27^{kiP1} degradation and Cks2 by other mechanisms.

In summary, the present invention demonstrates that aberrant expression of Cks1 in prostate tumor cells promotes tumorigenicity by promoting proliferation, anchorage-independent growth, and cellular migration activity, while aberrant expression of Cks2 may promote the tumorigenicity by protecting the cells from apoptosis. Together with the reports that Cks1 and Cks2 are not essential for somatic cell growth and tissue homeostasis, the results discussed herein suggest a novel strategy for prostatic cancer treatment without affecting normal somatic cells.

In one embodiment of the present invention, there is provided a method for treating prostate cancer in an individual in need of such treatment, comprising the step of: administering pharmaceutically effective amounts of a compound that downregulates the expression of the Cks1 gene, the Cks2 gene or both to the individual, thereby treating the prostate cancer in the individual. This method may further comprise administering pharmaceutically effective amounts of a chemotherapeutic drug. Such a chemotherapeutic drug may be administered concurrently or sequentially with the compound. Examples of the compound that downregulates expression of the Cks1 gene, the Cks2 gene or both may include but is not limited to a peptide nucleic acid (PNA) or RNA-mediated interference. In general, treatment may inhibit tumor growth, proliferation, angiogenesis, metastasis, or a combination thereof in the individual. Specifically, the downregulation of expression of Cks1 gene may inhibit proliferation, anchorage-independent growth, and migration activities of prostate tumor cells and the downregulation of expression of Cks2 gene may induce programmed cell death and inhibit tumorigenicity of prostate tumor cells.

In another embodiment of the present invention, there is provided a method for treating prostate cancer in an individual in need of such treatment, comprising the step of administering pharmaceutically effective amounts of a compound that downregulates the expression of the Cks1 gene product, the Cks2 gene product or both to the individual, thereby treating the prostate cancer in the individual. Such a method may further comprise administering pharmaceutically effective amounts of a chemotherapeutic drug. The chemotherapeutic drug may be administered concurrently or sequentially with the compound. Examples of the compound that downregulates expression of the Cks1 gene product, the Cks2 gene product or both may include but is not limited to an antibody, a Cks1 antisense RNA, a Cks2 antisense RNA or a small molecule inhibitor. Representative examples of Cks1 inhibitors are disclosed in United States Patent Application 20060089321.

In general, treatment may inhibit tumor growth, proliferation, angiogenesis, metastasis, or a combination thereof in the individual. Specifically, the downregulation of expression of the Cks1 gene product may inhibit proliferation, anchorage-independent growth, and migration activities of prostate tumor cells and the downregulation of expression of the Cks2 gene product may induce programmed cell death and inhibits tumorigenicity of prostate tumor cells.

In yet another embodiment, there is provided a method of diagnosing prostate cancer in an individual, comprising: obtaining a biological sample from the individual; and determining expression level of the Cks1 gene, the Cks1 gene product, the Cks2 gene, the Cks2 gene product or a combination thereof in the sample, wherein the overexpression of the Cks1 gene, the Cks1 gene product, the Cks2 gene, the Cks2 gene product or a combination thereof compared to expression level of Cks1 gene, Cks1 gene product, Cks2 gene, Cks2 gene product or a combination thereof in the sample from a control individual indicates that the individual has prostate cancer. This method may further comprise determining the presence of other markers characteristic of prostate cancer. Examples of the biological sample may include but is not limited to tumor tissue biopsy, fine needle aspiration biopsy, whole blood, serum, or plasma. Further, the expression of the Cks1 gene, the Cks2 gene or both may be determined by Northern blot, PCR, RT-PCR, dot blot or DNA microarray. Furthermore, the expression of Cks1 gene product, Cks2 gene product or both may be determined by Western blot, dot blot, ELISA, radioimmunoassay, flow cytometry, SELDI-TOF, mass spectrometry, protein array, tissue array, immunohistochemistry or other proteomic assays. The individual diagnosed using such a method may be one who is suspected of suffering from cancer or is at risk of developing cancer.

A chemotherapeutic drug may be administered concurrently or sequentially with the compound that downregulates expression of Cks1 gene or gene product and Cks2 gene or gene product as discussed herein. The effect of co-administration with the compound is to treat cancer at the same time. The compound described herein, the chemotherapeutic drug, or combination thereof can be administered independently, either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant. Dosage formulations of the compound described herein may comprise conventional non-toxic, physiologically or pharmaceutically acceptable carriers or vehicles suitable for the method of administration and are well known to an individual having ordinary skill in this art.

The compound described herein, the chemotherapeutic drug or combination thereof may be administered independently one or more times to achieve, maintain or improve upon a therapeutic effect. It is well within the skill of an artisan to determine dosage or whether a suitable dosage of either or both of the compound and chemotherapeutic drug comprises a single administered dose or multiple administered doses. An appropriate dosage depends on the subject's health, the inhibition of the tumor cell proliferation, the induction of programmed cell death and/or treatment of the cancer, the route of administration and the formulation used.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Animals

All animals were housed in the Program of Animal Resources at the Institute of Biosciences and Technology and were handled in accordance with the principles and procedures of the Guide for the Care and Use of Laboratory Animals. All experimental procedures were approved by the Institutional Animal Care and Use Committee. Genotypes of the mice were determined by PCR analyses as described (27-28). For tumorigenesis, 1×10⁶ cells were implanted subcutaneously in 0.1 ml MEM medium. The mice were observed carefully, and the palpable tumors were recorded daily. Tumors were harvested and histologically characterized as described (27-28). Tissues were fixed with 4% paraformaldehyde paraffin embedded. In situ hybridization was carried out with paraffin-embedded sections as described (29).

### Example 2

### cDNA Constructions

Full-length cDNAs for mouse Cks1 and Cks2 were RT-PCR amplified from mouse embryo RNA pools with primers mcks1-u: CTCCTGCAGAGCGATCATGTCGCACAAACA (SEQ ID NO: 1) and mcks1-d: CTTCTGCAGCTTCATTTCTTTGGTTTCTTG (SEQ ID NO: 2), and mcks2-u: TTTCTGCAGCGCGCCGGCAGGATGGCC (SEQ ID NO: 3) and mcks2-d: AGATMGCTTCATTTTTGTTGTTCTTTTGG (SEQ ID NO: 4), respectively. The cDNAs were cloned into the pBluescript SK vector for sequence verification and pEGFP-N3 vector for expression in mammalian cells (24).

The cDNA for rat Cks1-shRNA was constructed by annealing the primers pRcks1-t: GATCCGTCAGMGCATGTGAAATCTTCAAGAGAGATTTCACATGCTTCTGACT TTTTTACGCGTG (SEQ ID NO: 5) and pRcks1-b: AATTCACGCGTAAAAAAG TCAGAAGC ATGTGAAATCTCTCTTGAAGATTTCACATGCTTCTGACG (SEQ ID NO: 6); rat Cks2-shRNA by pRcks2-t: GATCCGCCAGAACCGCATATTCTTTTCAAGAGAAAGAATATGCGG TTCTGGCTTTTTTACGCGTG (SEQ ID NO: 7) and pRcks2-b: AATTCACGCGTAAAAAAGC CAGAACCGCAT ATTCTTTCTCTTGAAAAGAATATGCGGTT CTGGCG (SEQ ID NO: 8); mouse Cks1 shRNA by pMcks1-t: GATCCGGTCCACTATATGATCCATTTCAAGAGAATGG ATCATATAGTGGACCTTTTTTACGCGTG (SEQ ID NO: 9) and pMcks1-b: AATTCACGCGT AAAAAAGGTCCACTATATGATCCATTCTCTTGAAATGGATCATATAGTGGACCG (SEQ ID NO: 10) and mouse Cks2 shRNA by pMcks2-t: GATCCGTCCAACAGAGTCTAGGATTTCAA GAGAATCCTAGACTCTGTTGGACTTTTTTACGCGTG (SEQ ID NO: 11), and pMcks2-b: AA TTCACGCGTAAAAAAGTCCAACAGAGTCTAGGATTCTCTTGAAATCCTAGACTCTGTTGG ACG (SEQ ID NO: 12). The annealed oligonucleotides were cloned to the pSIREN-RetroQ-Tet vectors (Clontech, Mountain View, Calif.) and the sequences were verified.

### Example 3

### Gene Expression Analyses

Expression of Cks1 and Cks2 was analyzed by RT-PCR and RNase protection assay (RPA). Total RNA was extracted from tissues or cells with RNAzol (Ambion, San Antonio, Tex.). Reverse transcriptions were carried out with 1 mg RNA and 2.5 mg random hexamers with SuperScript reverse transcription kit (Invitrogen, Carlsbad, Calif.). PCR was carried out for Cks1 and Cks2 with the indicated primers for 30 cycles at 94 C, 55 C, and 72 C for 1 min each using the Taq DNA Polymerase (Promega, Madison, Wis.). Primers for rat Cks1 are rcks1-u: CTGGATCCGCAAATTCACACCATCCCTG (SEQ ID NO: 13) and rcks1-d: GTGATATCCTGATAGCGTGACCGTGTTG (SEQ ID NO: 14), and rat Cks2 are rcks2-u: CGTTTCCTTGTCCCGGTTT (SEQ ID NO: 15) and rcks2-d: TATGCGGTTCTGGCTCATGG (SEQ ID NO: 16), respectively. Primers for human Cks1 are hcks1-u: CTCCTGCAGAGCGATCATGTCGCACAAACA (SEQ ID NO: 17) and hcks1-d: CTTCTGCAGCTTCATTTCTTTGGTTTCTTG (SEQ ID NO: 18). Primers for human Cks2 are hcks2-u: TTTCTGCAGCGCGCCGGCAGGATGGCC (SEQ ID NO: 19) and hcks2-d: AGATAAGCTTCATTTGTTGTTCTTTTGG (SEQ ID NO: 20). The RT-PCR products were verified in sizes on a 2% agarose gel. For RPA, the RNA probe was transcribed and radiolabeled with −[³²P]UTP from the Cks1 and Cks2 cDNA templates with the Maxiscript Kit (Ambion, San Antonio, Tex.) (30). The labeled antisense RNA probe was hybridized with 25 g of total RNA at 68° C. for 11 minutes followed by incubation with RNase A1/T1 at 37° C. for 30 minutes. Protected fragments were separated on 5 percent polyacrylamide sequencing gels and the protected fragments were visualized by autoradiography.

### Example 4

### Cell Culture

The cells were maintained in RD media (50% RPMI, 50% DMEM) containing 5% fetal bovine serum (FBS). For proliferation assays, 10³ cells per well were inoculated to 24-well plates containing 1 ml of RD medium with 5%, or indicated concentrations of FBS. The cell numbers were counted daily. For soft agar growth assays, 1000 cells per well were mixed with 0.6% top agar at a ratio of 1:1 and inoculated to 6-well plates containing 1% base agar. The final serum concentration in the top and base agar was 5%. The cells were cultured for 3-4 weeks until the colonies were visible. The colonies were stained with Crystal Purple for observation and quantification. Data are mean and standard deviation (±SD) of three separate samples.

To establish stable cell lines expressing the tetracycline-controlled transcriptional suppressor (tTS), cells (5×10⁵) were transfected with the ptTS-Neo Vector (Clontech, Mountain View, Calif.) and selected for G418 resistant. The healthy colonies with the highest tetracycline inducible expression of the pSIREN-RetroQ-Tet-Luc reporter were picked for further transfection with pSIREN-RetroQ-Tet-Cks1/Cks2-shRNA plasmids. After being selectively cultured in hygromycin (200 mg/ml) media, the colonies were screening for the best knockdown efficiency induced by doxycycline at a concentration of mg/ml. RT-PCR analyses were employed for assessing the knockdown efficiency. For overexpression analyses, the stable transfectants were pooled to avoid clonal deviations. RT-PCR analyses were used to confirm expression of Cks1 and Cks2.

### Example 5

### Cell Migration Assay

Prior to the experiments, the cells were cultured in the presence doxycycline for over 48 hours to induce expression of shRNAs. The cells (2.5×10⁴) in 0.5 ml of serum-free RD medium were inoculated to gelatin-coated 6.5 mm Millicell inserts of 8 mm pore size (COSTA Corning N.Y.) placed in 12-well culture plates, which contain 10% FBS RD medium with or without doxycycline. After incubation at 37° C. for 12 hours, the cells migrated to the outer surface of the membrane were fixed, stained with hematoxylin, and counted under a microscope.

### Example 6

### Apoptosis Analysis

Cells (3×10⁵) cultured in 6-well dishes in 5% FBS/RD medium were treated with doxycycline for the indicated days to induce shRNA expression. The cells were then stained with the APOPercentage™ dye (Biocolor Ltd, Newtonabbey, Northern Ireland) for 30 minutes. The stained cells were visualized by microscopy, and the positive cells were quantified. For Annexin V staining, the cells (1-5×10⁵) were harvested 48 hours after the transfection and suspended in 500 ml binding buffer containing 1% Annexin V-Cy3. The cells were replated on 6-well dishes and the Annexin V-Cy3 stained cells were observed under a fluorescent microscope.

### Example 7

### Western Blotting

The cells (2×10⁵) in 6-well plates were cultured in 5% FBS for 24 hours and the expression of shRNA was induced for 48 hours. The cells were then lysed with 0.5% Triton-PBS. Aliquots containing 10 mg of protein were subjected to SDS-polyacrylamide gel electrophoreses (SDS-PAGE) on 3 gels, and were electroblotted to nylon membranes for Western blot analyses with the indicated antibodies.

For Akt and Bcl-xl staining, the membrane was cut into two pieces for staining with anti-Akt and anti-Bcl-xl antibodies individually. The other 2 membranes were stained with anti-Bad and anti-b-actin, respectively. The experiments were repeated for 3 times and the representative results were showed. The specifically bound antibodies were detected with the ECL-plus chemiluminescent detection reagents. Anti-p27^{kiP1}, anti-Bcl-xl, anti-Bad, and anti-b-actin antibodies were purchased from Santa Cruz Biotechnology Inc., (Santa Cruz, CA).

### Example 8

### Expression of Cks1 and Cks2 is Elevated in Prostate Cancer

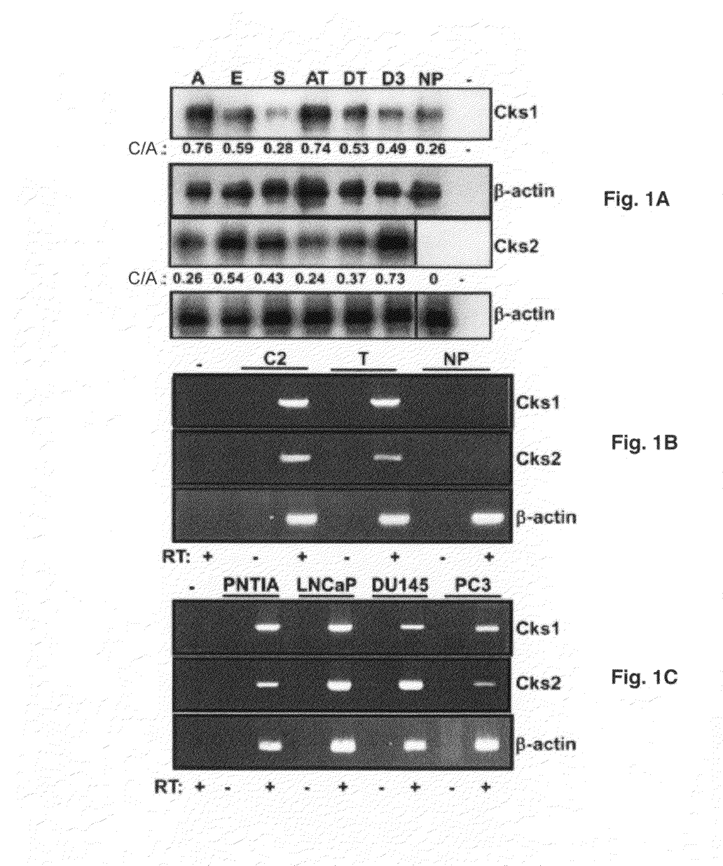

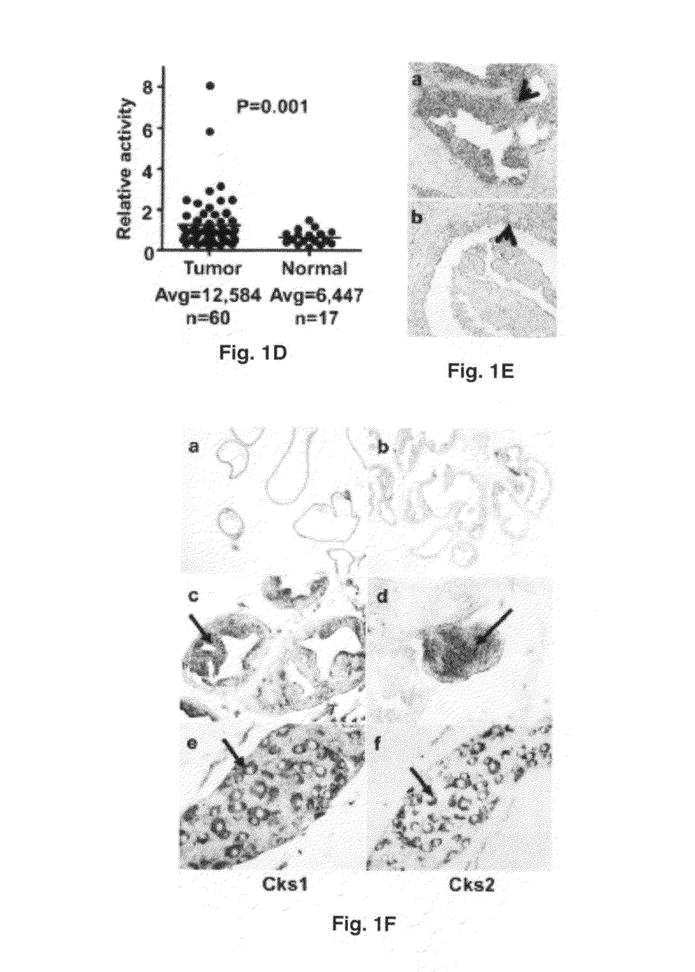

To determine whether expression of Cks1 and Cks2 was elevated in prostate tumors, RPA was employed to quantitatively analyze expression of Cks1 and Cks2 mRNA in rat prostates and the Dunning3327 (DT3) rat prostate tumors, which consist of androgen-dependent benign DT tumors and androgen-independent malignant AT tumors (31-32). The results showed that Cks1 was weakly expressed in normal rat prostates and the benign DT3 tumors; the expression was apparently stronger in the malignant AT tumors than in normal prostates and benign prostate tumors (FIG. 1A). The Cks2 expression in normal rat prostates was below the detection limit (FIG. 1A). Strong Cks2 expression was evident in benign DT3 and malignant AT tumors, as well as in cell lines derived from both tumors, including the DTE epithelial and DTS stromal cells derived from benign DT tumors, and AT3 cells from the malignant AT tumors (FIG. 1A).

To determine whether Cks1 and Cks2 were overexpressed in other prostate tumors, RT-PCR (30 cycles) was employed to assess Cks1 and Cks2 expression in the TRAMP autochthonous mouse prostate tumor model (28). Expression of Cks1 and Cks2 was apparent in TRAMP tumor tissues and the C2 cells derived from TRAMP tumors. Under the same condition, Cks expression was undetectable in adult mouse prostates (FIG. 1B).

To investigate whether aberrant expression of Cks1 and Cks2 was associated with human prostate cancers, RT-PCR analyses were employed to assess the expression of Cks1 and Cks2 in human prostate cell lines, including nontumorigenic prostate epithelial cell line PNTIA, low tumorigenic LNCaP, and highly tumorigenic DU-145 and PC3 cells (FIG. 1C). The results showed that expression of Cks1 and Cks2 mRNA was evident in all tested cell lines, including the T antigen-immortalized human prostate epithelial cells isolated from the normal prostate tissues adjacent to prostate tumors.

Furthermore, real-time RT-PCR analyses of cDNAs prepared from human prostate cancer and its peripheral normal tissues showed that a significant number of the tested tumors expressed Cks2 at a higher level than did the controls. Overall, relative to normal peripheral tissues, elevated expression of Cks2 was apparent in the tumor tissues (p<0.01), whereas the difference in Cks1 expression was not statistically significant between the tumor and normal peripheral zone tissues (FIG. 1D). In situ hybridization also demonstrated that expression of Cks2 was evident in lesion foci with high-grade prostate intraepithelial neoplasia (PIN) in human prostate (FIG. 1E panel a), whereas no Cks2 expression was detectable in the epithelium with normal tissue histology (FIG. 1D panel b).

To further investigate whether aberrant expression of Cks was associated with prostate tumorigenesis, prostate tissues were harvested from TRAMP mice and wildtype littermates at the age of 4-8 weeks. Tissue sections with relatively normal tissue structures, low to medium grade PIN, high grade PIN, and adenocarcinomas were selected for in situ hybridization analyses (FIG. 1F). The results confirmed that Cks1 and Cks2 expression in normal prostate was under the detection limit (panels a,b). The expression of Cks was also very low in foci with low grade PIN lesions, whereas the expression was apparent in tissues with lesions of high grade PIN (panels c and d, arrows), and adenocarcinomas (panels e and f, arrows).

### Example 9

### Knockdown of Cks1 and Cks2 Expression Inhibits Population Growth of Prostate Cancer Cells

To study the role of aberrantly expressed Cks1 and Cks2 in prostatic cancer cells, expression of Cks1 and Cks2 were knocked down in malignant AT3 cells by stably transfected with cDNAs encoding Cks1- or Cks2-shRNA cloned in a doxycycline-inducible vector. Real-time RT-PCR analyses revealed that expression of Cks1 and Cks2 in the cells was reduced to about 25% and 35% of the original levels in the presence of doxycycline inducers. To determine whether knockdown of Cks1 or Cks2 expression inhibited cell growth, population curves of the cells cultured in normal media containing 5% FBS were measured with and without the induction of Cks1- and Cks2-shRNA expression.

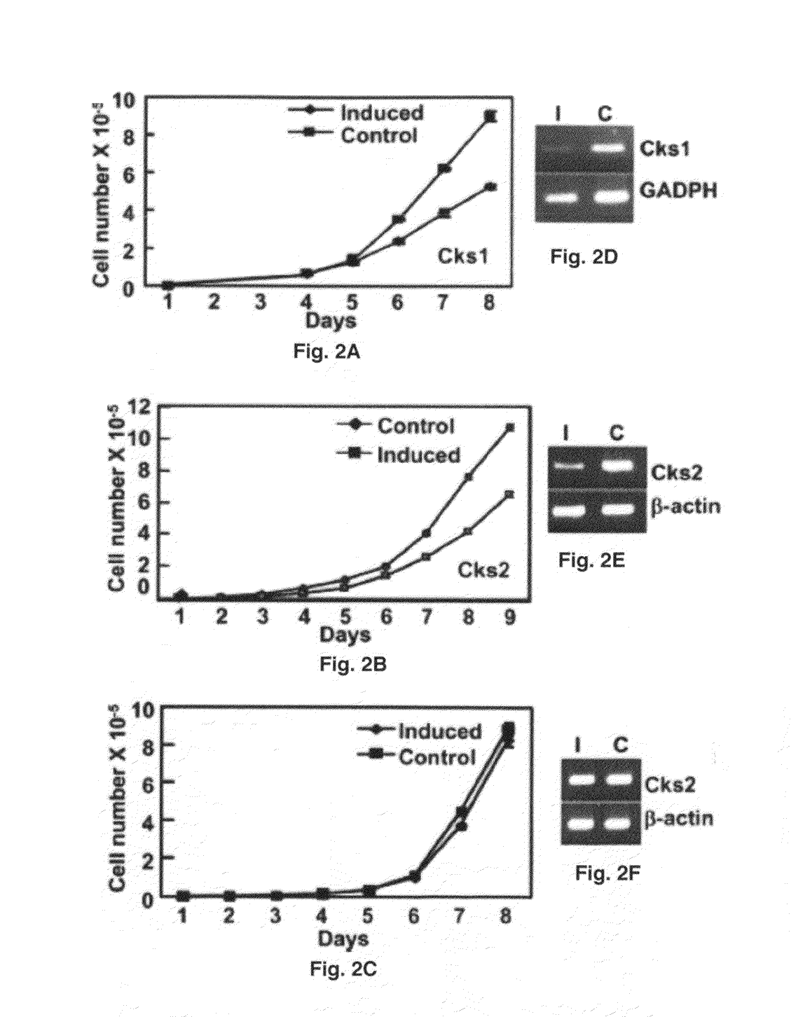

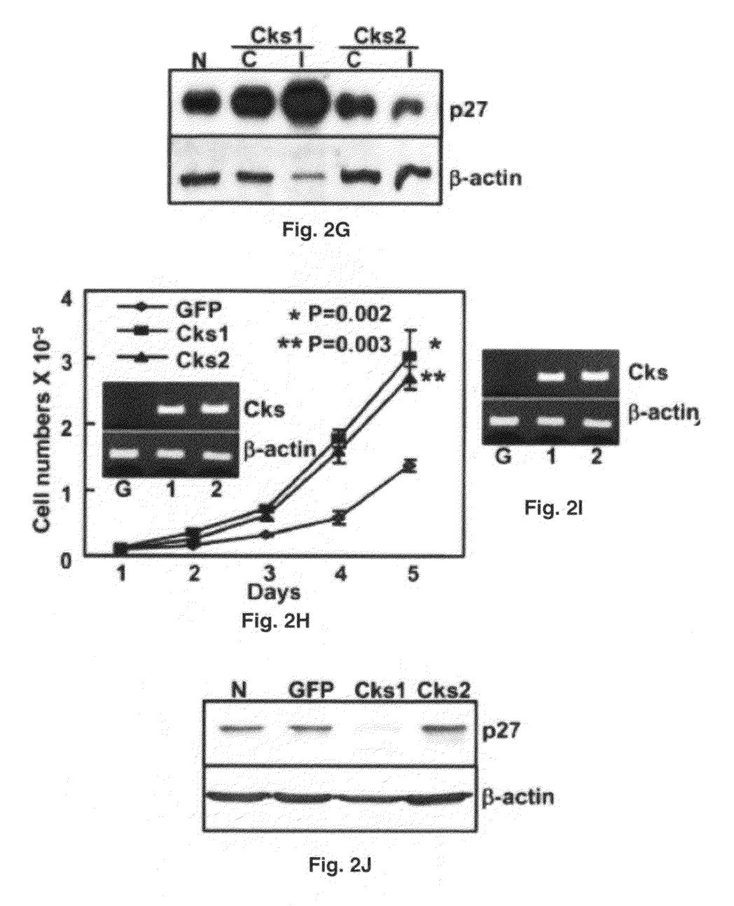

The results showed that Cks1 knockdown (FIGS. 2A-2F) reduced the population growth rate of the cells. Under the same condition, doxycycline treatment did not affect the growth rate of cells expressing stochastic shRNA and Cks2 shRNA. Yet, when the cells were cultured in suboptimal media, including low serum concentration or containing 50% medium conditioned by AT3 cells, the population doubling rate was apparently reduced by expression of Cks2-shRNA. Western analyses demonstrated that the protein abundance of p27^{kiP1} was elevated in Cks1, but not Cks2, knockdown cells (FIG. 2G), which was consistent with the finding that Cks1 triggers degradation of p27^{kiP1}. The results suggest Cks2 may be critical for cells to grow or survive in non-optimal conditions. Comparable to the cells expressing GFP alone or untransfected cells, DTE cells expressing either Cks1- or Cks2-GFP fusion proteins exhibited an accelerated population growth activity (FIGS. 2H-2I). Interestingly, only the cells overexpressing Cks1, but not Cks2, had reduced p27^{kiP1} abundance, which is a negative regulator of cell cycle progression (FIG. 2J). The results suggest that overexpressed Cks1 accelerates the population growth by directly promoting p27^{kiP1} degradation and Cks2 by other mechanisms.

### Example 10

### Knockdown of Expression of Cks2 Induces Programmed Cell Death

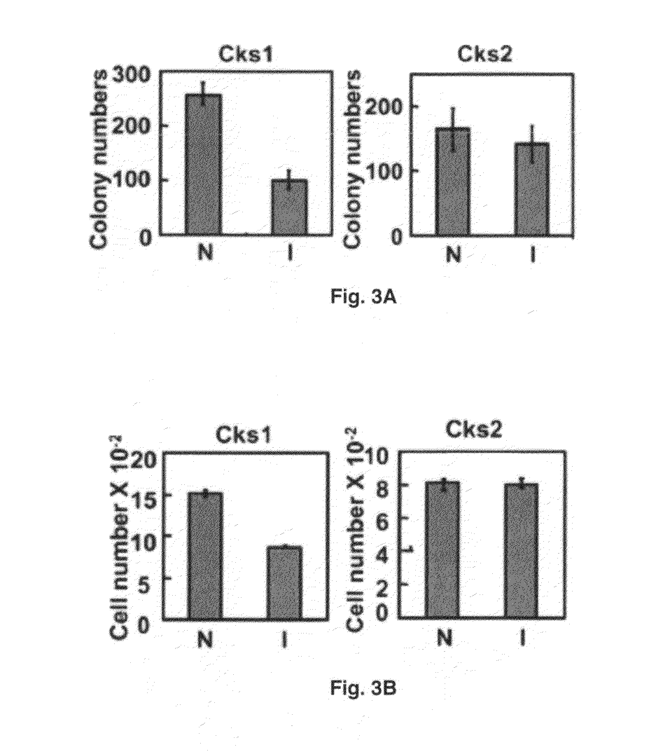

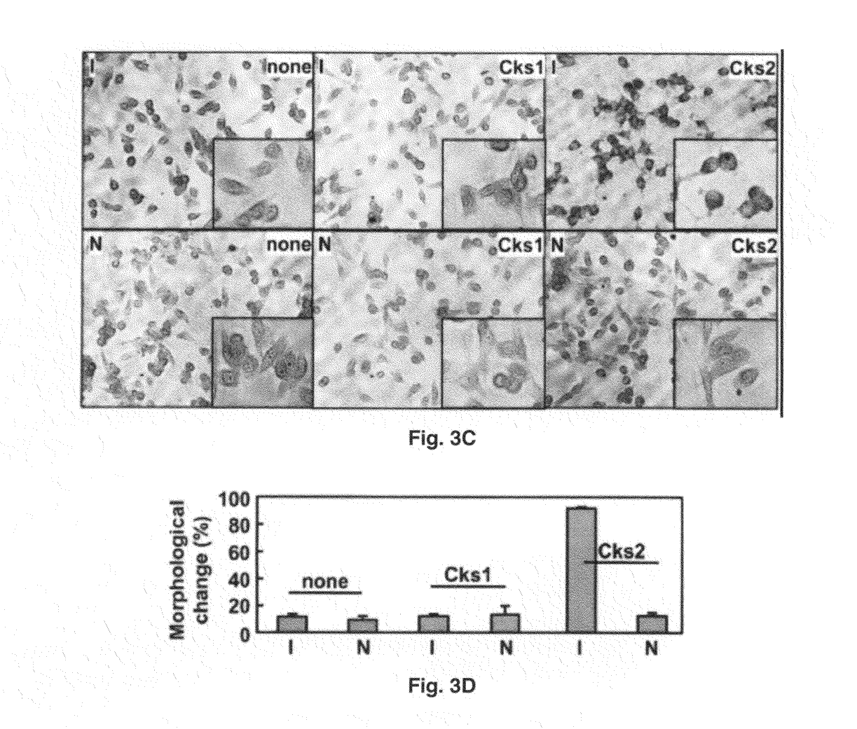

To further investigate the role of elevated expressed Cks1 or Cks2 in prostate tumor cells, the colony formation activity of AT3 cells stably transfected with the inducible shRNA constructs were assessed. It was apparent that the colony numbers were reduced by knockdown of Cks1 (FIG. 3A), suggesting that elevated expression of Cks1 in malignant AT3 cells contributed to the anchorage-independent growth activity. In contrast, knockdown of Cks2 expression only slightly reduced the colony number, suggestive that Cks2 might not be essential for the anchorage-independent growth activity. Furthermore, cell migration assay showed that knocking down Cks1, but not Cks2, expression in AT3 cells significantly reduced cell migration activity (FIG. 3B). Separated experiments revealed that force expression of Cks1 and Cks2 to benign DTE cells did not affect the anchorage-independent growth and cell migration activities.

Although knockdown of Cks2 expression did not severely inhibit migration and anchorage-independent growth in AT3 cells, it induced dramatic cell morphology change within 12 hours after the induction especially when the cells were cultured in a suboptimal culture medium that contained 50% of fresh medium and 50% of conditioned media. Most cells became rounded and loosely attached to the culture surface (FIGS. 3C,D), suggestive of programmed cell death. To test this possibility, the cells cultured in suboptimal conditions were stained with the APOPercentage™ dye 12-72 hours after the induction with doxycycline, which stained the apoptotic cells that lost the membrane potential.

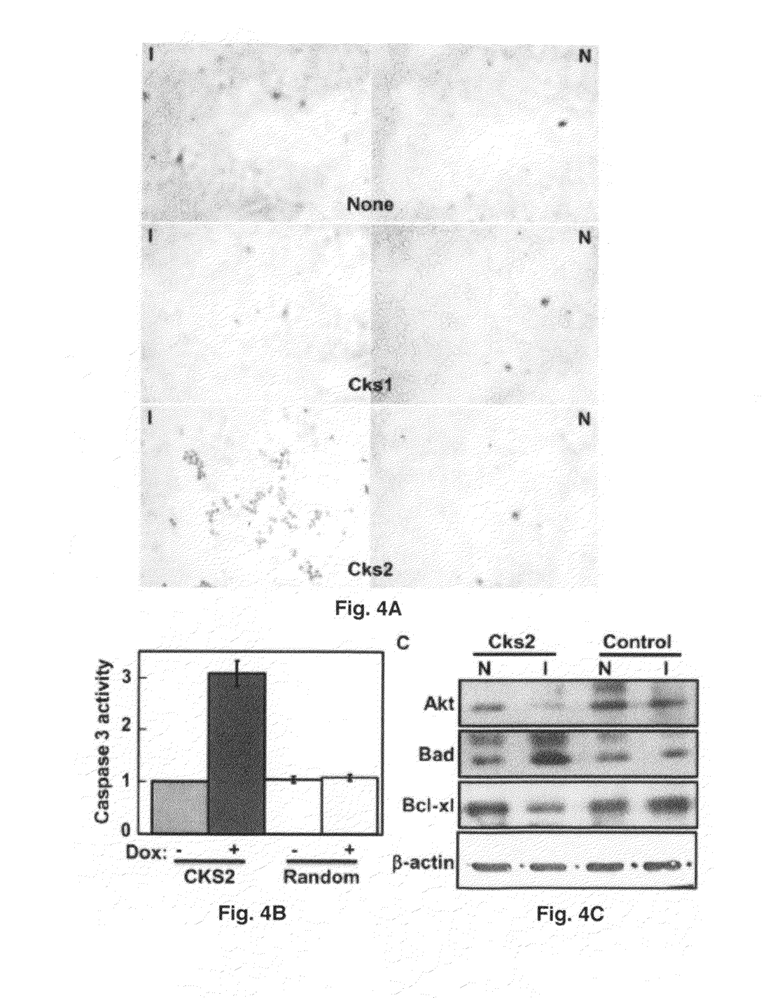

The results showed that knockdown of Cks2 expression induced significant cell death within 12 hours (FIG. 4A). In contrast, knockdown of Cks1 expression did not induce apoptosis under the same condition. Consistently, knockdown of Cks2 expression increased the caspase 3 activity by at least 2 fold (FIG. 4B). To further confirm this, Western blot analyses were carried out to assess the abundance of apoptosis markers at the protein levels. It was apparent that knockdown of Cks2 expression increased Bad and somewhat decreased Bcl-xl and Akt expression at the protein levels (FIG. 4C).

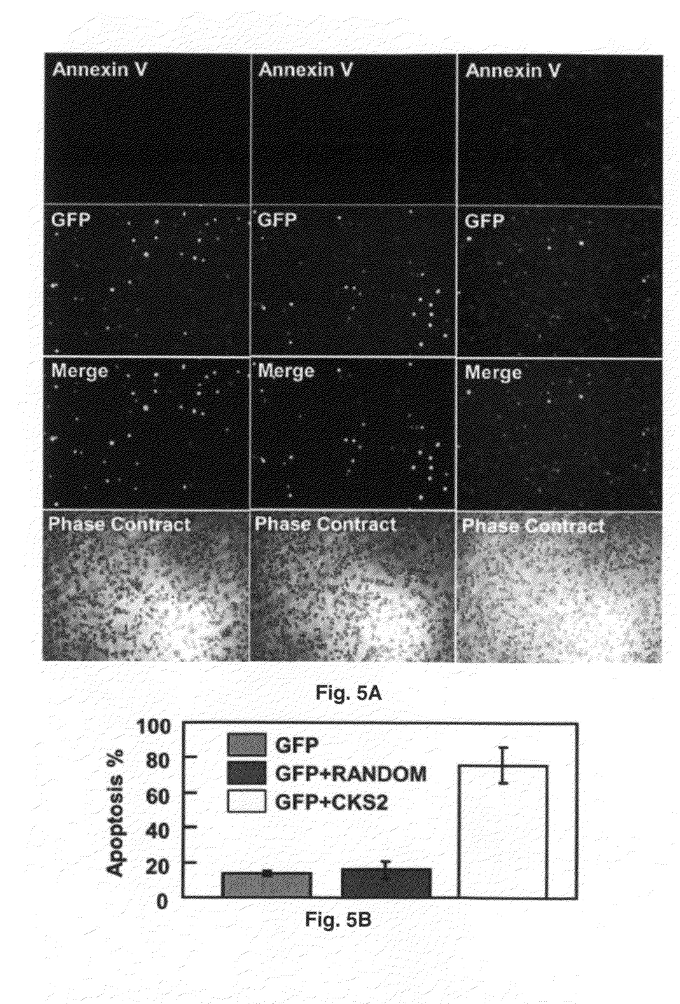

In order to determine whether knockdown of Cks2 expression also induced apoptosis in other prostate cancer cells, Cks2 shRNA was transiently expressed in mouse prostate tumor cells (C2) derived from TRAMP tumors (28). To monitor the transfected cells, the GFP was coexpressed in the cells. Since about 85% of the transfectants are cotransfected with more than one vector, the majority of the GFP expressing cells would also express Cks2 shRNA. If knocking down Cks2 expression induced apoptosis in C2 cells, the majority of GFP positive cells would undergo apoptosis. The cells were stained with Cy3 conjugated Annexin V that recognized phosphatidylserine exposed only in apoptotic cell surface. Fluorescent microscopy analyses showed that, indeed, at 48 hours after the transfection, about 80% of GFP-positive cells were stained with Annexin V-Cy3, suggestive of the programmed cell death (FIG. 5). It is noticeable that not every GFP positive cells underwent apoptosis, and not every apoptotic cells expressed GFP, which reflects the fact that not every GFP positive cells also expressed Cks2 shRNA, and vice versa. In contrast, coexpression of stochastic shRNA and GFP or GFP alone rarely induced apoptosis in cells (FIG. 5). Similarly, knockdown of Cks2 expression also induced apoptosis in human DU145 cells. Thus, these results support a role of aberrantly expressed Cks2 in protecting prostate cancer cells from undergoing apoptosis.

### Example 11

### Knockdown of Cks2 Expression Inhibits Tumorigenicity of Prostate Cancer Cells

The data that elevated Cks2 expression protected the cells from undergoing programmed cell death prompted an investigatation of whether knockdown of Cks2 expression inhibited tumorigenicity of the cells. TRAMP C2 cells stably transfected with the inducible Cks2-shRNA construct were implanted to synergetic C57 mice as described (33). Doxycycline was administrated through drinking water to induce expression of Cks2-shRNA.

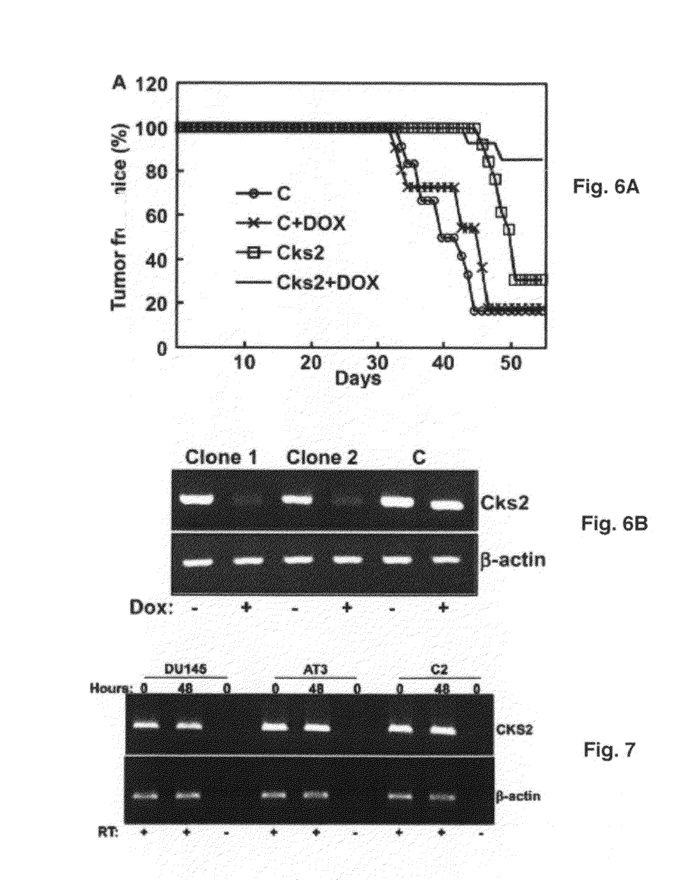

As expected, the control cells gave rise to tumors within 2-3 weeks and caused the death of host animals by the time of 30-45 days. In sharp contrast, only 14% animals implanted with the cells expressing Cks2-shRNA died by the time of 55 days (FIG. 6A). The results were confirmed by separated experiments with another individual clone. It is noticeable that the Cks2-shRNA transfectants were less tumorigenic than the control even in the absence of doxycycline inducers. RT-PCR analyses revealed that expression of Cks2 in the two individual transfectants was somewhat lower than the cells expressing stochastic shRNA even in the absence of doxycycline (FIG. 6B), suggestive of leaky expression of Cks2 shRNA. Thus, the reduction in Cks2 expression may inhibit tumorigenesis of the cells in the absence of doxycycline inducer.

The following references were cited herein:
1. Hayles et al., Mol Gen Genet 1986; 202:291-3.
2. Richardson et al., Genes Dev 1990; 4:1332-44.
3. Hadwiger et al., Mol Cell Biol 1989; 9:2034-41.
4. Spruck et al., Mol Cell 2001; 7:639-50.
5. Ganoth et al., Nat Cell Biol 2001; 3:321-4.
6. Urbanowicz-Kachnowicz et al., Int J Cancer 1999; 82:98-104.
7. Spruck et al., Science 2003; 300:647-50.
8. Kawakami et al., Oncol Rep 2006; 16:521-31.
9. Wong et al. Int J Cancer 2006; 118:2461-9.
10. Chow et al., Oncogene 2006; 25:310-6.
11. de Wit et al., Br J Cancer 2005; 92:2249-61.
12. Zirn et al., Oncogene 2005; 24:5246-51.
13. Lin et al., Breast Cancer Res 2007; 9:R25.
14. Uchikado et al., Int J Oncol 2006; 29:1337-47.
15. Rother et al., FEBS Lett 2007; 581:1166-72.
16. Kudo et al., Oral Oncol 2005; 41:105-16.
17. Ouellet et al., Oncogene 2005; 24:4672-87.
18. Shapira et al., Cancer 2005; 103:133646.
19. Slotky et al., Breast Cancer Res 2005; 7:R737-44.
20. Ouellet et al., Int J Cancer 2006; 119:599-607.
21. Li et al., Mod Pathol 2007; 20:497-507.
22. Hershko D D and Shapira M. Cancer 2006; 107:668-75.
23. Kitajima et al., Am J Pathol 2004; 165:2147-55.
24. Zhang et al., J Biol Chem 2004; 279:55348-54.
25. Shapira et al., J Urol 2006; 176:2285-9.
26. Lu X et al., Cell Cycle 2003; 2:59-63.
27. Jin et al., Cancer Res 2003; 63:8784-90.
28. Kaplan-Lefko et al., Prostate 2003; 55:219-37.
29. Lin et al., Development 2007; 134:723-34.
30. Wu et al., Cancer Res 2003; 63:493644.
31. McKeehan et al., Prog Nucleic Acid Res Mol Biol 1998; 59:135-76.
32. Feng et al., Cancer Res 1997; 57:5369-78.
33. Foster et al., Cancer Res 1997; 57:3325-30.
34. Brizuela et al., Embo J 1987; 6:3507-14.
35. Bloom J and Pagano M., Semin Cancer Biol 2003; 13:41-7.
36. Philipp-Staheli et al., Exp Cell Res 2001; 264:148-68.
37. Daniel et al., Lab Invest 2004; 84:588-96.
38. Itoh et al., J Biol Chem 2007; 282:390-6.
39. Li et al., Cancer Res 2006; 66:9986-94.
40. Nguyen et al., Genes Dev 2006; 20:1511-24.
41. Kawauchi et al., Nat Cell Biol 2006; 8:17-26.
42. Besson et al., Genes Dev 2004; 18:862-76.
43. Condeelis J and Segall J E. Nat Rev Cancer 2003; 3:921-30.
44. Baldassarre et al., Cancer Cell 2005; 7:51-63.
45. Galardi et al., J Biol Chem 2007; 282:23716-24.

Any patents or publications mentioned in this specification are indicative of the levels of those skilled in the art to which the invention pertains. Further, these patents and publications are incorporated by reference herein to the same extent as if each individual publication was specifically and individually incorporated by reference.

What is claimed is:

1. A method for treating prostate cancer in an individual in need of such treatment, comprising the step of:
administering a pharmaceutically effective amount of a compound that downregulates the expression of Cks1 gene, Cks2 gene or both to the individual, thereby treating the prostate cancer in the individual.

2. The method of claim 1, further comprising:
administering a pharmaceutically effective amount of a chemotherapeutic drug.

3. The method of claim 2, wherein said chemotherapeutic drug is administered concurrently or sequentially with the compound.

4. The method of claim 1, wherein said compound is a peptide nucleic acid (PNA) or RNA-mediated interference.

5. The method of claim 1, wherein said treatment inhibits tumor growth, proliferation, angiogenesis, metastasis, or a combination thereof in the individual.

6. The method of claim 1, wherein the downregulation of expression of Cks1 gene inhibits proliferation, anchorage-independent growth, and migration activities of prostate tumor cells.

7. The method of claim 1, wherein the downregulation of expression of Cks2 gene induces programmed cell death and inhibits tumorigenicity of prostate tumor cells.

8. A method for treating prostate cancer in an individual in need of such treatment, comprising the step of:
administering pharmaceutically effective amounts of a compound that downregulates the expression of Cks1 gene product, Cks2 gene product or both to the individual, thereby treating the prostate cancer in the individual.

9. The method of claim 7, further comprising:
administering pharmaceutically effective amounts of a chemotherapeutic drug.

10. The method of claim 9, wherein said chemotherapeutic drug is administered concurrently or sequentially with the compound.

11. The method of claim 7, wherein said compound is an antibody, a Cks1 antisense RNA, a Cks2 antisense RNA or a small molecule inhibitor.

12. The method of claim 7, wherein said treatment inhibits tumor growth, proliferation, angiogenesis, metastasis, or a combination thereof in the individual.

13. The method of claim 7, wherein the downregulation of expression of Cks1 gene product inhibits proliferation, anchorage-independent growth, and migration activities of prostate tumor cells.

14. The method of claim 7, wherein the downregulation of expression of Cks2 gene product induces programmed cell death and inhibits tumorigenicity of prostate tumor cells.

15. A method for diagnosing prostate cancer in an individual, comprising:
obtaining a biological sample from the individual; and
determining expression level of Cks1 gene, Cks1 gene product, Cks2 gene, Cks2 gene product or a combination thereof in the sample, wherein the overexpression Cks1 gene, Cks1 gene product, Cks2 gene, Cks2 gene product or a combination thereof compared to expression level of Cks1 gene, Cks1 gene product, Cks2 gene, Cks2 gene product or a combination thereof in the sample from a control individual indicates that the individual has prostate cancer.

16. The method of claim 15, further comprising:
determining the presence of other markers characteristic of prostate cancer.

17. The method of claim 15, wherein the biological sample is tumor tissue biopsy, fine needle aspiration biopsy, whole blood, serum, or plasma.

18. The method of claim 15, wherein the expression of Cks1 gene, Cks2 gene or both is determined by Northern blot, PCR, RT-PCR, dot blot or DNA microarray.

19. The method of claim 15, wherein the expression of Cks1 gene product, Cks2 gene product or both is determined by Western blot, dot blot, ELISA, radioimmunoassay, flow cytometry, SELDI-TOF, mass spectrometry, protein array, tissue array, immunohistochemistry or other proteomic assays.

20. The method of claim 15, wherein said individual is suspected of suffering from cancer or is at risk of developing cancer.
